(19) United States
(12) Patent Application Publication
(10) Pub. No.: US 2022/0008582 A1
(43) Pub. Date: Jan. 13, 2022

(54) UV EMITTER FOR DISINFECTING

(71) Applicant: Metacore Technologies LLC, Greenwich, CT (US)

(72) Inventor: Alexander Edward Gaynor, Greenwich, CT (US)

(21) Appl. No.: 17/354,546

(22) Filed: Jun. 22, 2021

Related U.S. Application Data
(60) Provisional application No. 63/042,123, filed on Jun. 22, 2020.

(51) Int. Cl. A61L 2/10

(52) U.S. Cl. CPC A61L 2/10; A61L 2202/122; A61L 2202/11

(57) ABSTRACT

A device utilizing one or more mechanically moving UVC emitters in conjunction with ozone gas treatment to disinfect various common items may be described herein. Disinfection may be achieved by mechanically following the surface of the object, at close range, with the UVC light. This may increase the intensity of the UVC light, which in turn, decreases the time required to reach sanitization. The mechanically moving UVC light may allow the device to disinfect an object thoroughly due to its ability to reposition the emitters. The system described herein may sanitize surfaces not in a direct line of sight of the UVC light. A small-scale system, described herein, may quickly disinfect objects that have been re-contaminated and/or where a full-scale sanitization system is impracticable. Additionally, a system aimed at reducing the cost and increasing the efficiency of frequently sanitizing objects may be described herein.

### CROSS-REFERENCE TO RELATED APPLICATIONS

This application claims the benefit of U.S. Provisional Application No. 63/042,123, filed on Jun. 22, 2020 the contents of which are incorporated herein by reference in their entirety.

### TECHNICAL FIELD

The application generally relates to systems and methods for disinfecting products or objects with electromagnetic radiation and ozone gas. More specifically the application relates to using ultraviolet light, specifically the UVC wavelength, in close range and angled directly at the object being disinfected.

### BACKGROUND

Disinfection is considered to be the primary mechanism for the inactivation/destruction of pathogenic organisms present on articles to prevent the spread of diseases to downstream users and the environment. It is important that items such as medical devices and tools be properly disinfected/sterilized prior to reuse.

In the past, attempts to sanitize objects generally included washing and cleansing an object and then packaging and/or wrapping the object, which normally took place in special clean processing facilities. However, it is not always feasible or desirable to set up significant special facilities to sanitize such objects to desirable levels. For example, it may be desirable to package and/or wrap a food product at a convenient location where no special facilities are normally available such as at an office, a home, or even outdoors. Similarly, it may be desirable to package and/or wrap a medical device or instrument with no special medical cleansing facility being available or desirable for sanitizing the medical instrument before a subsequent use.

In most circumstances after providing sanitizing agents and cleansing facility to help clean and sanitize a product or object, subsequent poor handling by personnel typically results in re-contamination prior to final packaging of the product or object. This poor handling creates serious contamination hazards and transfer of disease to users and consumers of the products and objects being packaged under such conditions. Most commonly, an expensive special handling and processing facility is required to provide a sanitizing and/or sterilizing effect to an object or product. For example, irradiation processing of object and products requires very specialized and expensive equipment that is not readily usable in most environments.

In medical applications, where medical equipment and instruments need to be sanitized, unfortunately, conventional specialized equipment must be used to sanitize and disinfect the equipment or instruments to a satisfactory level, or possibly sterilize as necessary, for further use. This specialized equipment is usually expensive and the process for sanitizing, disinfecting, and/or sterilizing, tends to be time consuming significantly impacting the costs of medical services and the commercial viability of medical businesses. Additionally, this specialized equipment and processing is normally not generally available in all but specialized environments.

Thus, there is a need for a small scale and inexpensive disinfecting system that can quickly and effectively disinfect objects.

### SUMMARY

Focused and mechanically moved Ultra-Violet C (UVC) light may be described herein as a method for disinfection. A device that utilizes one or more mechanically moving UVC emitters in conjunction with ozone gas treatment in order to disinfect various common items entering a household may be described herein. Disinfection may be achieved by mechanically following the surface of the object, at close range, with the UVC light. This may increase the intensity of the UVC light, which in turn, decreases the time required to reach sanitization. Additionally, the mechanically moving UVC light may allow the device to disinfect an object thoroughly due to its ability to reposition the emitters.

A system described herein may sanitize surfaces not in a direct line of sight of the UVC light. A small-scale system, described herein, may quickly disinfect objects that have been re-contaminated and/or where a full-scale sanitization system is impracticable. Additionally, a system aimed at reducing the cost and increasing the efficiency of frequently sanitizing objects may be described herein.

In an example, the system for disinfecting objects may include a housing, for receiving an object to be disinfected, and a plurality of tracks inside the housing. An array of diodes capable of emitting electromagnetic radiation may be attached to at least one of the tracks. An actuator may be configured to move the array of diodes along the tracks. A controller may activate the actuator to move the array of diodes along the track.

In another example, the system for disinfecting objects may include a bed for receiving an object to be disinfected. An actuator may be configured to move the bed. An array of diodes capable of emitting electromagnetic radiation may be positioned around the bed. A controller may activate the actuator to move the bed.

In another example, the system for disinfecting objects may include a controller, a housing, and an array of diodes. The array of diodes may emit electromagnetic radiation and may move inside the housing. The system may also include a sensor providing data regarding at least one surface of an object. The sensor may be in data communications with the controller. The controller may actuate the movement of the array of diodes based on the data regarding the surface of the object.

### BRIEF DESCRIPTION OF THE DRAWINGS

FIG. 1 is an example cross section of a disinfecting device with gas tubes;

FIG. 2 is an example of a disinfecting device with the lid open and gas tubes exposed;

FIG. 3 is an example cross section of a disinfecting device with a needle device;

FIG. 4 is an example cross section of a disinfecting device with the lid removed;

FIG. 5 is an example of a UV emitter mechanism;

FIG. 6 is an example cross section of a disinfecting device while in use;

FIG. 7 is an example of a disinfecting device without a housing;

FIG. 8 is an example embodiment of a UV emitter having two extendable prongs;

FIG. 9 is an example embodiment of a UV emitter having one extendable prong and a counterweight;

FIG. 10 is an example embodiment of a UV emitter having a rotatable cylinder; and

FIG. 11 is a schematic drawing of a system for disinfecting objects.

### DETAILED DESCRIPTION

Focused and mechanically moved UVC (Ultra-Violet) light may be described herein as a method for disinfection. The “C” frequency of the electromagnetic UV family has, amongst other things, germicidal effects. A device that utilizes a mechanically moving UV emitter in conjunction with ozone gas treatment in order to disinfect various common items entering a household may be described herein. Disinfection may be achieved by mechanically following the surface of the object, at close range, with the UVC light. This may exponentially increase the intensity of the UVC light, which in turn, may decrease the time required to reach sanitization.

A combination treatment using ozone gas to disinfect surfaces and volumes that are not in direct line of sight with the UV emitters may be described herein.

A device that utilizes a mechanically moving UV emitter in conjunction with ozone gas treatment in order to disinfect various items may be described herein. The device may disinfect various items for residential use, school use, nursing home use, retail use, salon/barber use, gym use, and the like. By using the UV light with ozone gas, the device may be able to disinfect both surfaces that are and are not directly in the line of sight with the UV light sources.

For example, common items entering a household may include, but are not limited to shopping bags, groceries, mail, mail parcels, mail/shipping packages, common kitchen items, infant care, children's toys, electronic devices, personal items, rental/refurbished items, animal care, and the like.

For example, common items for school use may include, but are not limited to school nurse equipment, college bookstores, retail items, athletic equipment, and the like.

For example, common items for nursing home use may include, but are not limited mail, packages, gifts, items from family, medical equipment, and the like.

For example, common items for retail use may include, but are not limited to items shared and handled by customers, including merchandise, pens, credit card machines, and the like.

For example, common items for gym use may include, but are not limited to dumbbells, straps, medicine balls, kettlebells, plates, and the like.

The UV emitters may allow for the device to effectively sanitize objects very quickly because it is able to shorten the distance between the UV emitter and the surface to be disinfected. The distance between the UV emitter and the object may be shortened by moving the UV emitter closer to the object or may be shortened by moving the object closer to the UV emitter. The UV emitters may be able to be moved and angled right up to the surface of any object to disinfect as quickly as possible.

The ozone treatment may be delivered by a device manipulating the atmospheric conditions inside an airtight chamber creating ozone from the present oxygen or ozone may be pumped directly into the object to be sanitized via a tube/needle apparatus.

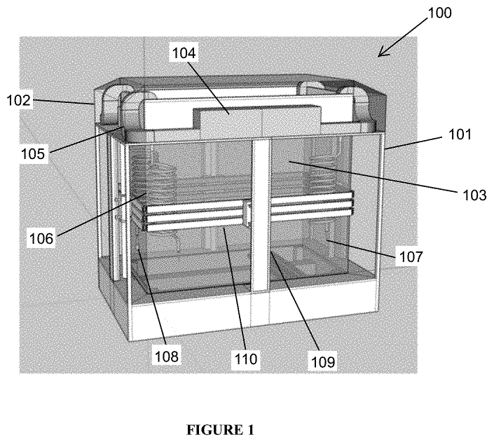

FIG. 1 is an example cross section of a disinfecting device with gas tubes. The disinfecting device 100 may include a housing 101 with a lid 102. The housing 101 may have an internal chamber 103 for receiving an object. The device 100, housing 101, and lid 102, may be rectangular, circular or any other shape.

The disinfecting device 100 may include an ozone generator 104 and tubing 105 for ozone direction located in the lid 102. Tubing 105 in the lid 102 may connect to flexible tubing 106 in the internal chamber 103, supplying a constant stream of ozone gas to the emitters 107, 108. The device 100 may have multiple ozone emitters 107, 108. A needle may be used for injecting ozone into a package, whereas a diffuser may be placed inside of a bag. A linear stage actuator 109 may be used to move the UV emitters 110 along an edge of an internal chamber 103.

In an example embodiment, the disinfecting device 100 may have a similar linear stage actuator 109 and UV emitters 110 combination along each side of the internal chamber 104 including the top and bottom.

The disinfecting device 100 may manipulate the atmospheric composition of the internal chamber 103 to sanitize the contents of the internal chamber 103. The disinfecting device 100 may use an ozone generator 104 and air purifiers on air that is circulated through the internal chamber 103 using tubing 105. This process may be used in conjunction with UV treatment from the UV emitters 110 to reach areas that the UV energy may not reach directly through line of sight.

For example, a stack of sports equipment that has small, shielded surfaces, such as helmets, may have areas out of sight of the UV energy and therefore the ozone may sanitize those areas more effectively.

The disinfecting device 100 may use a needle 107 or diffuser 108 to target specific areas, or an object, within the internal chamber 103 by supplying a concentration of ozone.

In one example embodiment, the disinfecting device 100 may manipulate the atmospheric composition of the entire internal chamber 103. The housing 101 and the lid 102 may form an airtight seal and the ozone generator 104 may fill the entire internal chamber 103 with ozone.

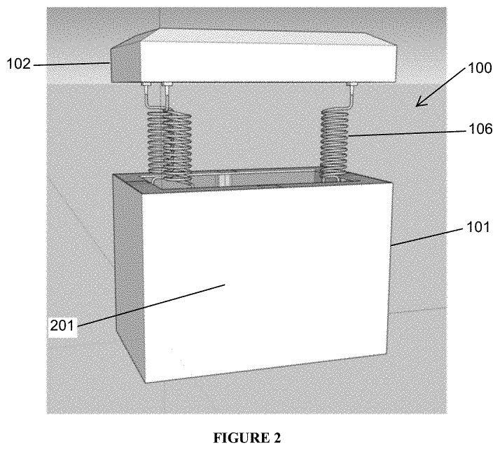

FIG. 2 is an example embodiment of a disinfecting device with the lid open and gas tubes exposed. The disinfecting device 100 may have an external enclosure 201 attached to the housing 101 and the lid 102. The external enclosure 201 may shield end users from harmful UV energy.

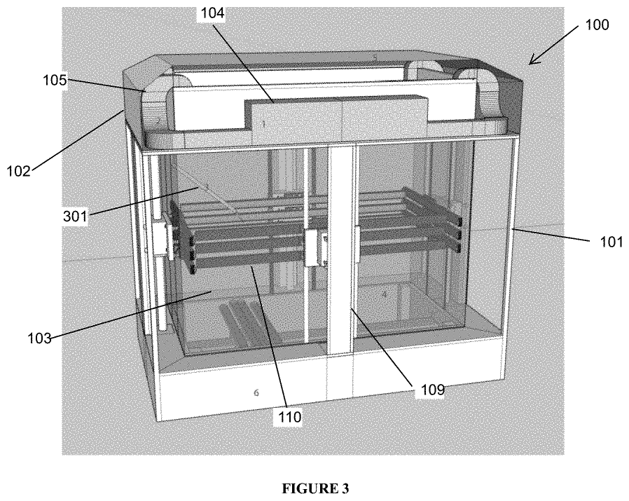

FIG. 3 is an example embodiment of a disinfecting device with a needle device. The disinfecting device 100 may be comprised of three major components: a housing 101, a lid 102, and an internal chamber 103.

The disinfecting device 100 may be compact. For example, the housing 101 may be 775 mm long, 477 mm wide, and 703 mm tall. The internal chamber 103 may fit an object(s) up to 572 mm long, 276 mm wide, and 408 mm tall.

The disinfecting device 100 may have an ozone treatment system using an ozone generator 104, tubing 105 to direct the ozone, and a needle device 301. For example, a user may place the needle device 301 through a piece of tape that has been placed over a gap in a package before operation. This may allow for ozone gas to flow directly into the package or other object, sanitizing the contents of the package. Additionally, an ozone sensor may be located in the lid 102 and may notify a controller when enough of the ozone has dissipated for the lid 102 to be opened. The controller may be an onboard or external computer, a smart phone, personal computer, or the like.

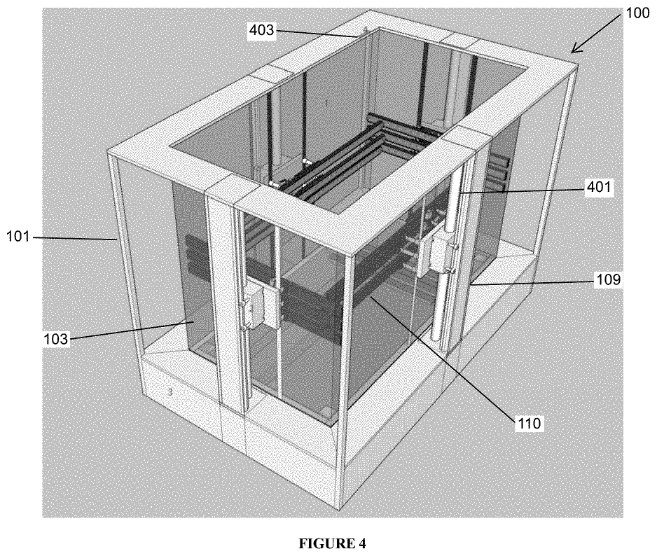

FIG. 4 is example cross section of the disinfecting device with the lid removed. The internal chamber 103 may use quartz glass, to transmit UVC radiation, for the bottom panel that supports the object being sanitized. The other panels of the internal chamber 103 may be made from standard acrylic plastic and may contain liner stage actuators 109 to move the UV emitters 110 through the internal chamber 103 on tracks 401. For example, the linear stage actuator 109 may be similar to those commonly found in a CNC machine. These actuators 109 may be supported by cutouts in the housing 101.

A magnetic contact switch 403 terminal may be attached to the housing 101 as well as the lid 102. The contact switch 403 may allow for the controller to prevent operation when the lid 102 is open.

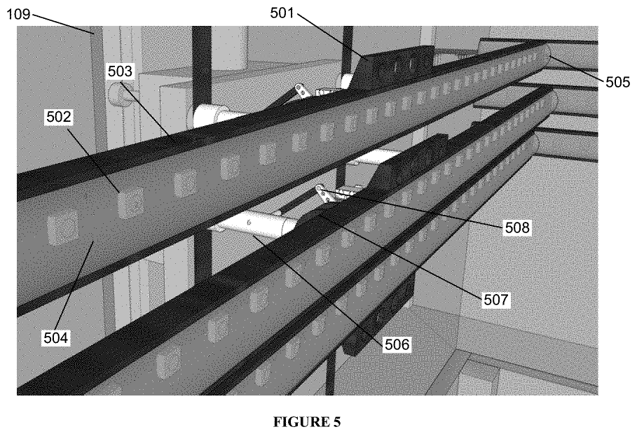

FIG. 5 is an example of a UV emitter mechanism. An individual emitter bar 501 may be comprised various sensors, an array of UVC emitting diodes 502, an external enclosure 503, reflector 504, and a focusing lens 505. The focusing lens 505 may be quartz glass. Attached to every individual emitter bar 501 may be a set of ultrasonic distance sensors and cameras. These sensors may feed data into the controller to help guide the emitter bar 501 along the surface of the object being disinfected.

The controller may control the position of the entire UV emitter bar 501 by using the linear stage actuator 109 it is attached to. Each individual emitter bar's 501 position may be controlled using the horizontal actuator 506 to move the bar 501 closer to the object. The angle of the emitter bar 501 may also be able to be controlled by the controller. Each emitter bar 501 may be connected to the horizontal actuator 506 using a hinge joint 507, a servo, and servo linkages 508. Using these methods for positioning, the controller may be able to accurately move the emitter bars 501 along various surface shapes.

The controller may utilize an algorithm to calculate the quickest way to sanitize an object given the type of object and its surface area. The algorithm may include several factors, for example, distance between the emitters and the object, strength of the electromagnetic radiation, the angle of incidence of the electromagnetic radiation, and other similar considerations. The controller may then control all of the internal mechanisms to execute the sanitization procedure.

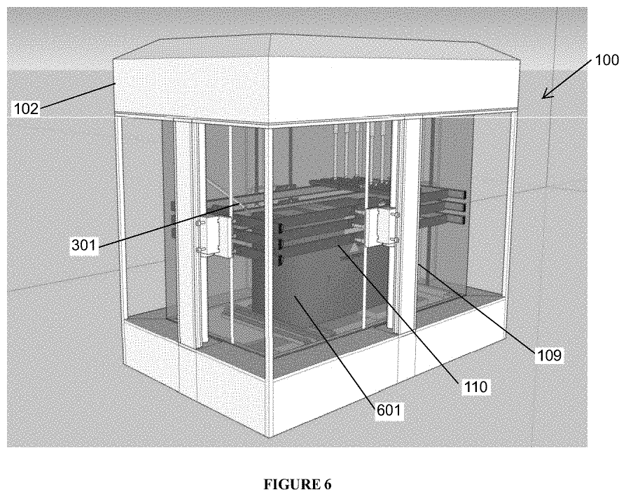

FIG. 6 is an example cross section of the disinfecting device while in use. An object 601, for example, a package, may be in the process of being sanitized. The needle device 301 may be attached to an object 601, pumping ozone gas directly into it, while the UV emitters 110 may be moving along the surfaces of the object 601. Once the process is complete, the user may be notified that their object 601 is sanitary and ready for use via a push notification from an accompanying smartphone app and/or an LED indicator on the lid 101. A UV emitter 110 may be located on every side of the disinfecting device 100.

Additionally, the disinfecting device 100 may have Internet of Things (IoT) connectivity with supporting software for end-users.

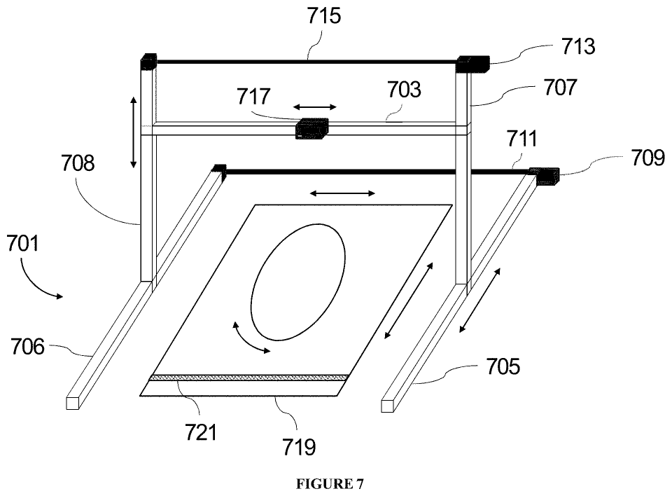

FIG. 7 is an example of a disinfecting device without a housing. The disinfecting device 100 may have a track and actuator system 701 with a single UV emitter device. The system 701 may be similar to a CNC machine or 3D printer and may be made up of a truss of tracks. In one embodiment the system 701 may consist of one x-axis track 703, two y-axis tracks 705, 706, and two z-axis tracks 707, 708. More or less tracks may be used depending on desired stability, range of motion, speed, and other similar considerations.

In one embodiment the system 701 may have a separate step motor 709, 713, 717 to control motion in each of the x, y, and z directions. The step motor 709, 713, 717 may be actuators, servomotors, or other motors.

In one embodiment where more than one track is in parallel with each other, a drive shaft may connect the parallel tracks with a single step motor. For example, the system 701 may have a first y-axis track 705 and a second y-axis track 706 in parallel and may have a y-axis drive shaft 711 connected to both tracks 705, 706 and the y-axis step motor 709. In a further example, the system 701 may have a first z-axis track 707 and a second z-axis track 708 in parallel and may have a z-axis drive shaft 715 connected to both tracks 707, 708 and the z-axis step motor 713. Thus, a single step motor may control motion in more than one track simultaneously.

The system 701 may be placed over a bed 719. The bed 719 may be made of quartz glass. A UV emitter 721 may be placed under the bed 719 and may move along the under surface of the bed 719, radiating UV light on any object placed on the bed 719.

In some embodiments the bed 719 may move back and forth and side to side. In another embodiment at least a portion of the bed 719 may rotate. In another embodiment the bed 719 may move and/or rotate around a stationary UV emitter. In another embodiment the bed 719 may move and/or rotate in addition to the UV emitter moving and/or rotating.

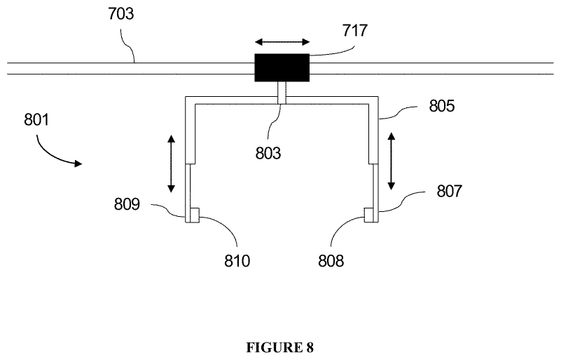

FIGS. 8-10 illustrate different embodiments of UV emitters.

FIG. 8 is an example embodiment of a UV emitter having two extendable prongs. The UV emitter device 801 may be attached to the x-axis step motor 717 by an axel 803. The axel 803 may be able to extend, retract and/or rotate.

In this example embodiment, the UV emitter device 801 may have a bar 805 attached to the axel 803. The bar 805 may have an extension pointing down on one, both, or neither end. A first extender 807 may be attached to the bar 805 and may be capable of extending and retracting. A first electromagnetic radiation device 808 may be attached to the first extender 807. The first electromagnetic radiation device 808 may be similar to the UV emitter bar 501 or may be any other array of diodes capable of emitting UCV energy.

The UV emitter device 801 may have a second extender 809 attached to the bar 805 capable of extending and retracting. A second electromagnetic radiation device 810 may be attached to the second extender 809. The second electromagnetic radiation device 810 may be similar to the UV emitter bar 501 or may be any other array of diodes capable of emitting UCV energy.

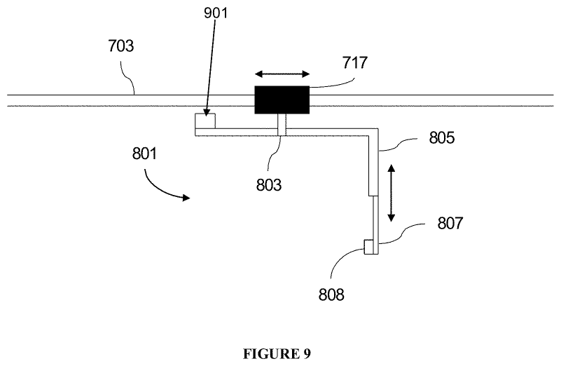

FIG. 9 is an example embodiment of a UV emitter having one extendable prong and a counterweight. In this example embodiment, the UV emitter device 801 may only have a first extender 807 attached to the bar 805. The bar 805 may have a counterweight 901 attached to the bar 805 opposite the first extender 807.

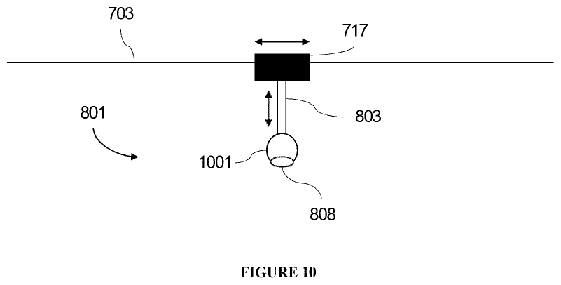

FIG. 10 is an example embodiment of a UV emitter having a rotatable cylinder. In this example embodiment, the UV emitter device 801 may have a cylinder 1001 attached to the axel 803. The cylinder 1001 may rotate close to 360 degrees around the cylinder's 1001 central axis. The first electromagnetic radiation device 808 may be attached to the cylinder 1001.

The UV emitter device 801 may be various shapes and configurations depending on the intended use.

In one embodiment the system 701 and the UV emitter device 801 may be enclosed in a device 100 as described above. The system 701 may be used in place of, or in conjunction with, the linear stage actuator 109. The UV emitter device 801 may be used in place of, or in conjunction with, the UV emitter 110.

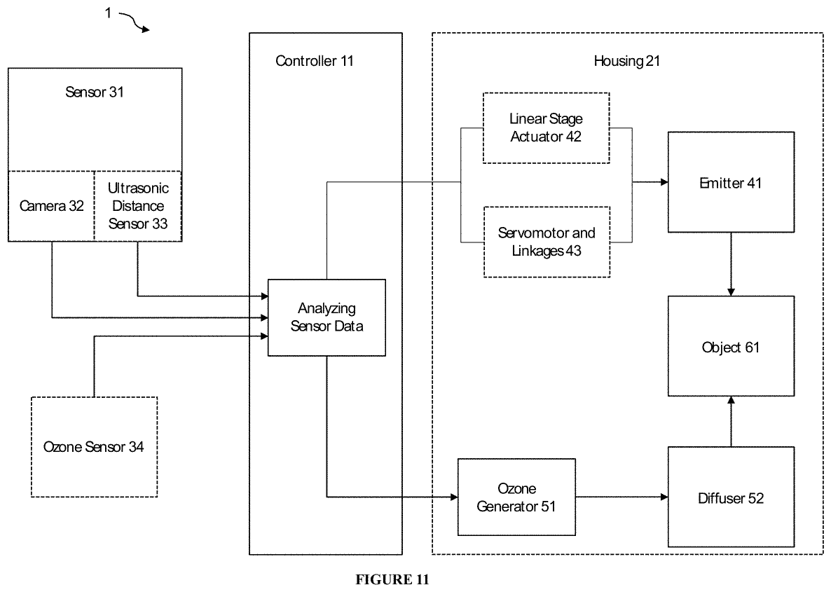

FIG. 11 is a schematic drawing of a system for disinfecting objects. The disinfecting system 1 may include a controller 11, a housing 21, a sensor 31, and an emitter 41. The controller 11 may be a computer, smart device, or other similar computing device. The housing 21 may be a protective shielding from UV energy and may be airtight.

The sensor 31 may be a camera 32, an ultrasonic distance sensor 33, or any other sensors capable of measuring distance between objects and surfaces, or any combination of the above.

The emitter 41 may be an array of diodes capable of emitting electromagnetic energy or any other emitter capable of disinfecting an object. The emitter 41 may have movement capabilities and may be connected to a linear stage actuator 42 and/or a servomotor and linkages 43, as well as any other means for performing computer numeric controlled movements. The servomotor and linkages 43 may be capable of extensions, retraction, rotation, or angling attached devices.

The disinfecting system 1 may also include an ozone sensor 34, an ozone generator 51, and a diffuser 52. The ozone sensor 34 may be any gas sensor capable of measuring the concentration of gasses in the ambient atmosphere. The ozone generator 51 may be a machine that converts air in the atmosphere into ozone, a tank of ozone, or other means for supplying ozone. The diffuser 52 may be an area diffuser, a targeted needle diffuser, or other means for pumping gas to an area.

The controller 11 may receive data from one or more of the sensors 31, ozone sensor 34, or other data source. The controller 11 may analyze the data to determine the optimal path for following the surface of an object 61 to achieve a desired level of sanitation. The controller 11 may control the movement of the emitter 41 via the linear stage actuator 42, servomotor and linkages 43 or other attached means of movement, to guide the emitter 41 along the surface of the object 61.

The controller 11 may activate the ozone generator 51 supplying ozone to the diffuser 52. The controller 11 may activate the ozone generator 51 until a desired amount of ozone reaches the object 61 to achieve the desired level of sanitation.

The housing 21 may contain the emitter 41 and attached movement mechanisms, the ozone generator 51, the diffuser 52, and the object 61. The housing 21 may protect end users from harmful UV energy, ozone gas, and other means utilized for sanitization.

Although the invention has been illustrated and described herein with reference to specific embodiments and examples thereof, it will be readily apparent to those of ordinary skill in the art that other embodiments and examples may perform similar functions and/or achieve user experiences. All such equivalent embodiments and examples are within the spirit and scope of the present invention, are contemplated thereby, and are intended to be covered by the disclosure.

In compliance with the statute, the present teachings have been described in language more or less specific as to structural and methodical features. It is to be understood, however, that the present teachings are not limited to the specific features shown and described, since the systems and methods herein disclosed comprise preferred forms of putting the present teachings into effect. The present disclosure is to be considered as an example of the invention and is not intended to limit the invention to a specific embodiment illustrated by the figures above or description below.

For purposes of explanation and not limitation, specific details are set forth such as particular architectures, interfaces, techniques, etc. in order to provide a thorough understanding. In other instances, detailed descriptions of well-known devices, circuits, and methods are omitted so as not to obscure the description with unnecessary detail.

Generally, all terms used are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The steps of any method disclosed herein do not have to be performed in the exact order disclosed, unless explicitly stated. The use of “first”, “second,” etc. for different features/components of the present disclosure are only intended to distinguish the features/components from other similar features/components and not to impart any order or hierarchy to the features/components. As used herein, the term “and/or” includes any and all combinations of one or more of the associated listed items. As used herein, the term “application” is intended to be interchangeable with the term “invention”, unless context clearly indicates otherwise.

While the present teachings have been described above in terms of specific embodiments, it is to be understood that they are not limited to these disclosed embodiments. Many modifications and other embodiments will come to mind to those skilled in the art to which this pertains, and which are intended to be and are covered by this disclosure. It is intended that the scope of the present teachings should be determined by proper interpretation and construction of the disclosure and its legal equivalents, as understood by those of skill in the art relying upon the disclosure in this specification and the attached drawings. In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification should be read with the understanding that such combinations are entirely within the scope of the invention.

What is claimed is:

1. A system for disinfecting objects comprising:
a housing configured to receive an object;
a plurality of tracks inside the housing;
an array of diodes capable of emitting electromagnetic radiation attached to at least one of the plurality of tracks;
an actuator configured to move the array along the tracks; and
a controller activating the actuators.

2. The system of claim 1, wherein the array of diodes emits UVC frequency radiation.

3. The system of claim 1, wherein the array of diodes has a quartz glass focusing lens.

4. The system of claim 1, wherein the plurality of tracks are configured to form a frame capable of at least vertical and horizontal movement within the housing

5. The system of claim 1, further comprising: a servomotor attached to the array of diodes configured to angle, rotate, extend, and retract the array of diodes.

6. The system of claim 5, further comprising:
a proximity sensor measuring a distance from the array of diodes and the object.

7. The system of claim 6, wherein the controller receives the distance from the proximity sensor and activates the actuators and the servomotor according to the received data.

8. The system of claim 1, further comprising:
a gas supplier, injecting a sanitizing gas into the housing.

9. The system of claim 8, further comprising:
a sensor measuring the concentration of sanitizing gas in the housing.

10. The system of claim 8, wherein the housing is airtight, and the housing is filled with ozone.

11. The system of claim 8, further comprising:
a needle apparatus connected to the gas supplier, wherein the needle is inserted into the object and ozone is pumped directly into the object.

12. A system for disinfecting objects comprising:
a bed configured to receive an object;
an actuator configured to move the bed;
an array of diodes capable of emitting electromagnetic radiation are positioned around the bed; and
a controller activating the actuator.

13. The system of claim 12, further comprising:
a plurality of tracks, wherein the array of diodes is attached to at least one of the plurality of tracks; and
a second actuator, activated by the controller, configured to move the array of diodes along the tracks.

14. The system of claim 12, wherein at least a portion of the bed is configured to rotate.

15. A system for disinfecting objects comprising:
a controller;
a housing;
an array of diodes, capable of emitting electromagnetic radiation, and capable of moving inside the housing;
a sensor, providing data regarding at least one surface of an object, in data communication with said controller; and
the controller actuating the movement of the array of diodes based on the data regarding the surface of the object.

16. The system of claim 15, further comprising:
a liner stage actuator and a servomotor with linkages providing movement capabilities to the array of diodes.

17. The system of claim 15, wherein the sensor also provides data regarding distance between the object and the array of diodes.

18. The system of claim 15, further comprising:
an ozone generator;
a diffuser; and
an ozone sensor capable of measuring a concentration of ozone;
wherein the controller activates the ozone generator supplying ozone to the diffuser based on the concentration of ozone.
